(19) United States
(12) Patent Application Publication
(10) Pub. No.: US 2016/0151469 A1
(43) Pub. Date: Jun. 2, 2016

(54) IMMUNE MODULATION BY TLR ACTIVATION FOR TREATMENT OF FILOVIRUS INFECTIONS INCLUDING EBOLA

(71) Applicant: Regen BioPharma, Inc., La Mesa, CA (US)

(72) Inventor: Thomas Ichim, San Diego, CA (US)

(21) Appl. No.: 14/954,902

(22) Filed: Nov. 30, 2015

Related U.S. Application Data
(60) Provisional application No. 62/085,636, filed on Nov. 30, 2014.

(51) Int. Cl. A61K 39/00; A61K 45/06

(52) U.S. Cl. CPC A61K 39/00; A61K 45/06; A61K 2039/58; A61K 2039/57

(57) ABSTRACT

Means and compositions of matter are disclosed for stimulation of innate immunity in controlling, substantially reducing, and/or clearing filoviral infections including Margburg and Ebola virus. In one embodiment an activator of dendritic cells (DC) is provided to replicate a state similar to one found in patients that significantly overcome filoviral infections. In one particular embodiment the HMGB1-derived peptide SAFFLFCSE or derivatives thereof are administered in a pharmacologically acceptable formulation. Efficacy may be augmented by administration of agents that increase monocyte numbers, which are thereafter stimulating to differentiate along the DC pathway by filoviral infection, or by administration of flt-3 ligand. Alternatively GM-CSF may be administered. Naturally derived compounds such as plant based lectins are also utilized to stimulate DC maturation through activation of receptors such as toll like receptors (TLR).

### CROSS-REFERENCE TO RELATED APPLICATIONS

This application claims the benefit of U.S. Provisional Application No. 62/085,636, filed Nov. 30, 2014, which is hereby incorporated in its entirety including all tables, figures, and claims.

### FIELD OF THE INVENTION

The invention pertains to the field of immune modulatory agents and their use for immunological enhancement for the treatment of viral infections. More specifically, the invention pertains to the field of innate immune stimulation through administration of agents capable of stimulating toll like receptors. More specifically, the invention relates to the use of peptides and toll like receptor agonists in the treatment of filoviral infections.

### BACKGROUND OF THE INVENTION

Ebola is one of the most pathogenic viruses known to man, with no FDA approved treatment and a mortality rate reaching up to 90%. Filamentous in shape, virus enters target cells through host Niemann-Pick C1 receptor where it lytically replicates its signal stranded negative sense RNA genome which encodes seven proteins. These can be categorized as: a) Surface glycoprotein (GP) which is found on viral surface and involved in cellular entry; b) The matrix protein VP40 which stabilizes the viral structure and is critical for budding; and c) The nucleocapside proteins, which comprise of nucleoprotein (N), L protein, VP24, VP30, and VP35.

Ebola viruses derive their name from the Ebola River in Zaire, attributed to the origins of the first Ebola outbreak in 1976 [1-3]. Along with Marburg viruses, Ebola viruses belongs to the Filoviridae family [4], which are all single-stranded, negative sense RNA viruses characterized by extreme hemorrhagic fever [5]. There are 5 species of Ebola viruses that are currently known, which are: Zaire (EBOV-Z), Sudan (EBOV-S), Cote-d′Ivoire (EBOV-C), Bundibugyo (EBOV-B), and Reston (EBOV-R). Of these, the EBOV-R is highly lethal in non-human primates but not in humans [6]. The EBOV-C initiated only one infection in humans that was not lethal [7]. EBOV-Z, EBOV-S, and EBOV-B are all highly lethal to humans, causing approximately 25-90% fatality in humans [8].

The original outbreaks of Ebola occurred almost simultaneously: between June to November 1976 in southern Sudan affecting 284 patients with a 53% mortality [9]; and in Northern Zaire between September and October 1976 affecting 318 patients with 88% mortality [10]. The causative agents were different and subsequently designated EBOV-S and EBOV-Z, respectively. The next outbreak occurred in 1989 in a primate research facility in Reston, United States, where cynomolgus monkeys displayed an infectious hemorrhagic fever. Fortunately the virus did not cause pathology in humans, although animal caretakers were found to possess antibodies to the Ebola [11, 12]. This is where the EBOV-R was identified. The monkeys were found to originate in the Philippines, where the EBOV-R was also found to infect pigs [13]. In 1994 a scientist studying an outbreak of hemorrhagic fever in chimpanzees in the West African country of Cote d′Ivoire was infected with what was identified as a new strain of Ebola, EBOV-C. The patient recovered subsequent to supportive therapy [14]. EBOV-B was first identified in the Bundibugyo District of Uganda infecting 55 patients. EBOV-B seems the least pathogenic of the EBOV-S and EBOV-Z strain in that morality was 44% [15]. The current 2014 Ebola outbreak that originated in West Africa belongs to the EBOV-Z strain [16-18].

### DESCRIPTION OF THE INVENTION

When practicing present invention it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To allow for the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

“antigen-presenting cells” or “APCs” are used to refer to autologous cells that express MHC Class I and/or Class II molecules that present antigens to T cells. Examples of antigen-presenting cells include, e.g., professional or non-professional antigen processing and presenting cells. Examples of professional APCs include, e.g., B cells, whole spleen cells, monocytes, macrophages, dendritic cells, fibroblasts or non-fractionated peripheral blood mononuclear cells (PMBC). Examples of hematopoietic APCs include dendritic cells, B cells and macrophages. Of course, it is understood that one of skill in the art will recognize that other antigen-presenting cells may be useful in the invention and that the invention is not limited to the exemplary cell types described herein. APCs may be “loaded” with an antigen that is pulsed, or loaded, with antigenic peptide or recombinant peptide derived from one or more antigens. In one embodiment, a peptide is the antigen and is generally antigenic fragment capable of inducing an immune response that is characterized by the activation of helper T cells, cytolytic T lymphocytes (cytolytic T cells or CTLs) that are directed against a malignancy or infection by a mammal. In one, embodiment the peptide includes one or more peptide fragments of an antigen that are presented by class I MHC or class II MHC molecules. The skilled artisan will recognize that peptides or protein fragments that are one or more fragments of other antigens may used with the present invention and that the invention is not limited to the exemplary peptides, tumor cells, cell clones, cell lines, cell supernatants, cell membranes, and/or antigens that are described herein.

“blood tissue” refers to cells suspended in or in contact with plasma.

“bone marrow cell” refers to any cell originating from the interior of bones.

“CD80,” “CD86,” “CD11c, “CD85” and similar terms refer to cell surface molecules present on leukocyte cells through a nomenclature protocol maintained by Human Cell Differentiation Molecules (www.hedm.org: Paris, France).

“dendritic cell” or “DC” refer to all DCs useful in the present invention, that is, DC is various stages of differentiation, maturation and/or activation. In one embodiment of the present invention, the dendritic cells and responding T cells are derived from healthy volunteers. In another embodiment, the dendritic cells and T cells are derived from patients with cancer or other forms of tumor disease. In yet another embodiment, dendritic cells are used for either autologous or allogeneic application.

“effective amount” refers to a quantity of an antigen or epitope that is sufficient to induce or amplify an immune response against a viral antigen.

“vaccine” refers to compositions that affect the course of the disease by causing an effect on cells of the adaptive immune response, namely, B cells and/or T cells. The effect of vaccines can include, for example, induction of cell mediated immunity or alteration of the response of the T cell to its antigen.

“immunologically effective” refers to an amount of antigen and antigen presenting cells loaded with one or more heat-shocked and/or killed tumor cells that elicit a change in the immune response to prevent or treat a viral infection. The amount of antigen-loaded and/or antigen-loaded APCs inserted or reinserted into the patient will vary between individuals depending on many factors. For example, different doses may be required for an effective immune response in a human with various viral infections, the genetic background of the individual and the type and strain of the virus.

“contacted” and “exposed”, when applied to an antigen and APC, are used herein to describe the process by which an antigen is placed in direct juxtaposition with the APC. To achieve antigen presentation by the APC, the antigen is provided in an amount effective to “prime” the APCs to express antigen-loaded MHC class I and/or class II antigens on the cell surface.

“therapeutically effective amount” refers to the amount of antigen-loaded APCs that, when administered to an animal in combination, is effective to kill virally infected cells within the animal. The methods and compositions of the present invention are equally suitable for killing a virally infected cell or cells both in vitro and in vivo. When the cells to be killed are located within an animal, the present invention may be used in conjunction or as part of a course of treatment that may also include one or more anti-viral agentst, e.g., chemical, irradiation, X-rays, UV-irradiation, microwaves, electronic emissions, and the like. The skilled artisan will recognize that the present invention may be used in conjunction with therapeutically effective amount of pharmaceutical composition such as existing antiviral compounds. However, the present invention includes live cells that are going to activate other immune cells that may be affected by the DNA damaging agent. As such, any chemical and/or other course of treatment will generally be timed to maximize the adaptive immune response while at the same time aiding to kill as many cancer cells as possible.

“antigen-loaded dendritic cells,” “antigen-pulsed dendritic cells” and the like refer to DCs that have been contacted with an antigen, in this case, virally infected cells that have been heat-shocked or untreated, or viral components themselves. Often, dendritic cells require a few hours, or up to a day, to process the antigen for presentation to naive and memory T-cells. It may be desirable to pulse the DC with antigen again after a day or two in order to enhance the uptake and processing of the antigen and/or provide one or more cytokines that will change the level of maturing of the DC. Once a DC has engulfed the antigen (e.g., pre-processed heat-shocked and/or killed cancer cells), it is termed an “antigen-primed DC”. Antigen-priming can be seen in DCs by immunostaining with, e.g., an antibody to the specific cancer cells used for pulsing. An antigen-loaded or pulsed DC population may be washed, concentrated, and infused directly into the patient as a type of vaccine or treatment against the pathogen or tumor cells from which the antigen originated. Generally, antigen-loaded DC are expected to interact with naive and/or memory T-lymphocytes in vivo, thus causing them to recognize and destroy cells displaying the antigen on their surfaces. In one embodiment, the antigen-loaded DC may even interact with T cells in vitro prior to reintroduction into a patient. The skilled artisan will know how to optimize the number of antigen-loaded DC per infusion, the number and the timing of infusions. For example, it will be common to infuse a patient with 1-2 million antigen-pulsed cells per infusion, but fewer cells may also induce the desired immune response.

The antigen-loaded DCs may be co-cultured with T-lymphocytes to produce antigen-specific T-cells. As used herein, the term “antigen-specific T-cells” refers to T-cells that proliferate upon exposure to the antigen-loaded APCs of the present invention, as well as to develop the ability to attack cells having the specific antigen on their surfaces. Such T-cells, e.g., cytotoxic T-cells, lyse target cells by a number of methods, e.g., releasing toxic enzymes such as granzymes and perforin onto the surface of the target cells or by effecting the entrance of these lytic enzymes into the target cell interior. Generally, cytotoxic T-cells express CD8 on their cell surface. T-cells that express the CD4 antigen CD4, commonly known as “helper” T-cells, can also help promote specific cytotoxic activity and may also be activated by the antigen-loaded APCs of the present invention. In certain embodiments, the cancer cells, the APCs and even the T-cells can be derived from the same donor whose MNC yielded the DC, which can be the patient or an HLA—or obtained from the individual patient that is going to be treated. Alternatively, the cancer cells, the APCs and/or the T-cells can be allogeneic.

The invention provides means of inducing an anti-viral response in a mammal, comprising the steps of initially “priming” the mammal by administering an agent that causes local accumulation of antigen presenting cells. Subsequently, a tumor antigen is administered in the local area where said agents causing accumulation of antigen presenting cells is administered. A time period is allowed to pass to allow for said antigen presenting cells to traffic to the lymph nodes. Subsequently a maturation signal, or a plurality of maturation signals are administered to enhance the ability of said antigen presenting cell to activate adaptive immunity. In some embodiments of the invention activators of adaptive immunity are concurrently given, as well as inhibitors of the tumor derived inhibitors are administered to derepress the immune system.

Culture of dendritic cells is well known in the art, for example, U.S. Pat. No. 6,936,468, issued to Robbins, et al., for the use of tolerogenic dendritic cells for enhancing tolerogenicity in a host and methods for making the same. Although the current invention aims to reduce tolerogenesis, the essential means of dendritic cell generation are disclosed in the patent. U.S. Pat. No. 6,734,014, issued to Hwu, et al., for methods and compositions for transforming dendritic cells and activating T cells. Briefly, recombinant dendritic cells are made by transforming a stem cell and differentiating the stem cell into a dendritic cell. The resulting dendritic cell is said to be an antigen presenting cell which activates T cells against MHC class I-antigen targets. Antigens for use in dendritic cell loading are taught in, e.g., U.S. Pat. No. 6,602,709, issued to Albert, et al. This patent teaches methods for use of apoptotic cells to deliver antigen to dendritic cells for induction or tolerization of T cells. The methods and compositions are said to be useful for delivering antigens to dendritic cells that are useful for inducing antigen-specific cytotoxic T lymphocytes and T helper cells. The disclosure includes assays for evaluating the activity of cytotoxic T lymphocytes. The antigens targeted to dendritic cells are apoptotic cells that may also be modified to express non-native antigens for presentation to the dendritic cells. The dendritic cells are said to be primed by the apoptotic cells (and fragments thereof) capable of processing and presenting the processed antigen and inducing cytotoxic T lymphocyte activity or may also be used in vaccine therapies. U.S. Pat. No. 6,455,299, issued to Steinman, et al., teaches methods of use for viral vectors to deliver antigen to dendritic cells. Methods and compositions are said to be useful for delivering antigens to dendritic cells, which are then useful for inducing T antigen specific cytotoxic T lymphocytes. The disclosure provides assays for evaluating the activity of cytotoxic T lymphocytes. Antigens are provided to dendritic cells using a viral vector such as influenza virus that may be modified to express non-native antigens for presentation to the dendritic cells. The dendritic cells are infected with the vector and are said to be capable of presenting the antigen and inducing cytotoxic T lymphocyte activity or may also be used as vaccines.

In one embodiment of the invention a patient suffering from Ebola is administered a peptide comprising the amino acids SAFFLFCSE or various peptides or peptide derivatives isolated from the protein HMGB1 that are capable of stimulating DC. Said peptide administration may be performed with the intention of local DC activation, or through systemic administration. The mode of administration for the peptide, or derivatives thereof, will vary accordingly to, for example, the type of subject, age, body weight, symptoms, therapeutic efficacy, method of administration and period of administration. For example, a single dose of the agent of the present invention containing the above-indicated effective dose of the peptide or the derivatives thereof in one embodiment may be orally administered from one to several times per day, or may be parenterally administered from one to several times per day. Alternatively, the peptide or derivatives thereof may be continuously administered intravenously for a period ranging from one hour to 24 hours per day, or may be continuously administered locally for a period ranging from one day to three months. When the peptide or derivatives thereof is administered, it may be used as a solid or liquid preparation for oral administration, or it may be used as an injection for parenteral administration, as an external preparation, as a gel. Injections of the peptide or derivatives thereof for parenteral administration encompass solutions, suspensions, emulsions, and solid injections which are dissolved or suspended in a solution at the time of use. An injection may be used after dissolving, suspending or emulsifying one or more active ingredient in a solvent. Examples of solvents that may be used include distilled water for injection, physiological saline, vegetable oils, propylene glycol, polyethylene glycol, alcohols such as ethanol, and combinations thereof. Such injections may also include stabilizers, solubilizers (e.g., glutamic acid, aspartic acid, Polysorbate 80 (registered trademark)), suspending agents, emulsifiers, analgesics, buffering agents and preservatives. These may be sterilized in the final step, or production and preparation may be carried out by aseptic manipulation. Alternatively, a sterile solid preparation, such a lyophilized product, may be produced, and this may be used by dissolution in distilled water for injection or some other solvent which is either sterilized before use or is aseptic.

In cases where an antigen peptide (such as VP35 from Ebola virus) and/or another drug is used in addition to the immunomodulatory peptides, these may both be used as ingredients of the agent of the present invention and administered in the form of a combination preparation obtained by combining both ingredients within a single preparation, or some or all of the antigen peptide and/or other drug may take a form which is administered as a separate preparation from the agent of the present invention. In cases where the antigen peptide and/or other drug takes a form which is administered as a separate preparation from the agent of the present invention, such preparations may be administered at the same time as the agent of the present invention or may be administered with a time interval. When administered with a time difference, the agent of the present invention, antigen peptide and other drugs are not subject to any particular limitation in the order of administration thereof, and may be administered in any order.

It is known in the art that patients who succumb to Ebola infection present with high concentrations of IL-10 [19]. Stimulation of immune cells, with particular reference to DC by TLR activators such as peptides derived from HMGB1 cause augmentation of IL-12 release by DC and suppression of IL-10 [20]. Accordingly, in the current invention upregulation of Th1 immunity is sought with downregulation of Th2 immunity.

### REFERENCES

1. Gonzalez, J. P., [Ebola, a tranquil river in the heart of Africa]. Sante, 1995. 5(3): p. 145-6.

2. Johnson, K. M., et al., Isolation and partial characterisation of a new virus causing acute haemorrhagic fever in Zaire. Lancet, 1977. 1(8011): p. 569-71.

3. Bowen, E. T., et al., Viral haemorrhagic fever in southern Sudan and northern Zaire. Preliminary studies on the aetiological agent. Lancet, 1977. 1(8011): p. 571-3.

4. Kuhn, J. H., et al., Proposal for a revised taxonomy of the family Filoviridae: classification, names of taxa and viruses, and virus abbreviations. Arch Virol, 2010. 155(12): p. 2083-103.

5. Tukei, P. M., Threat of Marburg and Ebola viral haemorrhagic fevers in Africa. East Afr Med J, 1996. 73(1): p. 27-31.

6. Morikawa, S., M. Saijo, and I. Kurane, Current knowledge on lower virulence of Reston Ebola virus (in French: Connaissances actuelles sur la moindre virulence du virus Ebola Reston). Comp Immunol Microbiol Infect Dis, 2007. 30(5-6): p. 391-8.

7. Le Guenno, B., et al., Isolation and partial characterisation of a new strain of Ebola virus. Lancet, 1995. 345(8960): p. 1271-4.

8. Feldmann, H. and T. W. Geisbert, Ebola haemorrhagic fever. Lancet, 2011. 377(9768): p. 849-62.

9. Ebola haemorrhagic fever in Sudan, 1976. Report of a WHO/International Study Team. Bull World Health Organ, 1978. 56(2): p. 247-70.

10. Ebola haemorrhagic fever in Zaire, 1976. Bull World Health Organ, 1978. 56(2): p. 271-93.

11. Jahrling, P. B., et al., Preliminary report: isolation of Ebola virus from monkeys imported to USA. Lancet, 1990. 335(8688): p. 502-5.

12. Centers for Disease, C., Update: evidence of filovirus infection in an animal caretaker in a research/service facility. MMWR Morb Mortal Wkly Rep, 1990. 39(17): p. 296-7.

13. Sayama, Y., et al., A seroepidemiologic study of Reston ebolavirus in swine in the Philippines. BMC Vet Res, 2012. 8: p. 82.

14. Formenty, P., et al., Human infection due to Ebola virus, subtype Cote d′Ivoire: clinical and biologic presentation. J Infect Dis, 1999. 179 Suppl 1: p. S48-53.

15. MacNeil, A., et al., Proportion of deaths and clinical features in Bundibugyo Ebola virus infection, Uganda. Emerg Infect Dis, 2010. 16(12): p. 1969-72.

16. Maganga, G. D., et al., Ebola Virus Disease in the Democratic Republic of Congo. N Engl J Med, 2014.

17. Gatherer, D., The 2014 Ebola virus disease outbreak in West Africa. J Gen Virol, 2014. 95(Pt 8): p. 1619-24.

18. Dixon, M. G., et al., Ebola viral disease outbreak—West Africa, 2014. MMWR Morb Mortal Wkly Rep, 2014. 63(25): p. 548-51.

19. Villinger, F., et al., Markedly elevated levels of interferon (IFN)-gamma, IFN-alpha, interleukin (IL)-2, IL-10, and tumor necrosis factor-alpha associated with fatal Ebola virus infection. J Infect Dis, 1999. 179 Suppl 1: p. S188-91.

20. Saenz, R., et al., TLR4-dependent activation of dendritic cells by an HMGB1-derived peptide adjuvant. J Transl Med, 2014. 12: p. 211.

1. A method of treating a patient suffering from a filoviral infection comprising administering to said patient a therapeutically effective amount an immune modulator capable of stimulating dendritic cell (DC) maturation.

2. The method of claim 1, wherein said immune modulatory is selected from a group comprising of: a) BCG; b) imiqimod; c) beta-glucan; d) hsp65; e) hsp90; f) HMGB-1; g) lipopolysaccharide; h) Pam3CSK4; i) Poly I: Poly C; j) Flagellin; k) MALP-2; l) lmidazoquinoline; m) Resiquimod; n) CpG oligonucleotides; o) zymosan; p) peptidoglycan; q) lipoteichoic acid; r) lipoprotein from gram-positive bacteria; s) lipoarabinomannan from mycobacteria; t) Polyadenylic-polyuridylic acid; u) monophosphoryl lipid A; v) single stranded RNA; w) double stranded RNA; x) 852A; y) rintatolimod; z) Gardiquimod; and aa) lipopolysaccharide peptides.

3. The method of claim 2, wherein said immune modulator is comprised of the amino acids SAFFLFCSE.

4. The method of claim 1, wherein an antioxidant is administered together with an immune modulatory at an amount sufficient to reduce inflammatory mediators secreted by filovirus infected cells, in order to augment efficacy of said immune modulator.

5. The method of claim 4, wherein said antioxidant is selected from a group comprising of: a) ascorbic acid and derivatives thereof; b) alpha tocopherol and derivatives thereof; c) rutin; d) quercetin; e) allopurinol; f) hesperidin; g) lycopene; h) resveratrol; i) tetrahydrocurcumin; j) rosmarinic acid; k) Ellagic acid; l) chlorogenic acid; m) oleuropein; n) alpha-lipoic acid; o) glutathione; p) polyphenols; q) pycnogenol; r) retinoic acid; s) ACE Inhibitory Dipeptide Met-Tyr; t) recombinant allogeneic superoxide dismutase; u) xenogenic superoxide dismutase; and v) superoxide dismutase.

6. The method of claim 1, wherein an agent is administered prior to, or concurrent with, said immune modulatory, said agent capable of increasing the number of DC progenitors, or DC in circulation.

7. The method of claim 6, wherein said agent capable of augmenting said DC or DC progenitors in circulation is selected from a group comprising of: a) G-CSF; b) GM-CSF; c) IL-4; d) flt-3 ligand; and e) M-CSF.

8. The method of claim 1, wherein said immune modulator is comprised of DPNAPKRPPSAFFLX.sub.1X.sub.2X.sub.3X.sub.4 or a derivative thereof, Wherein when X1 is alanine (A), glycine (G), or valine (V) then X2 is C, X3 is S and X4 is E; Wherein when X2 is alanine (A), glycine (G), or valine (V) then X1 is F, X3 is S and X4 is E; Wherein when X3 is alanine (A), glycine (G), or valine (V) then X1 is F, X2 is C and X4 is E; or Wherein when X4 is alanine (A), glycine (G), or valine (V) then X1 is F, X2 is C and X3 is S.

9. The method of claim 8, wherein DPNAPKRPPSAFFLX.sub.1X.sub.2X.sub.3X.sub.4 has the amino acid sequence: a. DPNAPKRPPSAFFLX.sub.1CSE, b. DPNAPKRPPSAFFLFX.sub.1SE, c. DPNAPKRPPSAFFLFCX.sub.1E, or Wherein X.sub.1 is alanine (A), glycine (G), or valine (V).

10. The method of claim 1, wherein DPNAPKRPPSAFFLX.sub.1X.sub.2X.sub.3X.sub.4 is further mutated so that F at amino acid positions 12 and 13 is changed to S.

11. The method of claim 8, wherein derivative is a fragment of DPNAPKRPPSAFFLX.sub.1X.sub.2X.sub.3X.sub.4 having the sequence RPPSAFFLX.sub.1X.sub.2X.sub.3X.sub.4, Wherein when X1 is alanine (A), glycine (G), or valine (V) then X2 is C, X3 is S and X4 is E; Wherein when X2 is alanine (A), glycine (G), or valine (V) then X1 is F, X3 is S and X4 is E; Wherein when X3 is alanine (A), glycine (G), or valine (V) then X1 is F, X2 is C and X4 is E; or Wherein when X4 is alanine (A), glycine (G), or valine (V) then X1 is F, X2 is C and X3 is S.

12. The method of claim 11, wherein RPPSAFFLX.sub.1X.sub.2X.sub.3X.sub.4 has the amino acid sequence: a. RPPSAFFLX.sub.1CSE, b. RPPSAFFLFX.sub.1SE, c. RPPSAFFLFCX.sub.1E, or d. RPPSAFFLFCSX.sub.1, Wherein X.sub.1 is alanine (A), glycine (G), or valine (V).

13. The method of claim 11, wherein RPPSAFFLX.sub.1X.sub.2X.sub.3X.sub.4 is further mutated so that F at amino acid positions 6 and 7 is changed to S.

14. The method of claim 11, wherein the derivative is a fragment of DPNAPKRPPSAFFLX.sub.1X.sub.2X.sub.3X.sub.4 having the amino acid sequence SAFFLX.sub.1X.sub.2X.sub.3X.sub.4, Wherein when X1 is alanine (A), glycine (G), or valine (V) then X2 is C, X3 is S and X4 is E; Wherein when X2 is alanine (A), glycine (G), or valine (V) then X1 is F, X3 is S and X4 is E; Wherein when X3 is alanine (A), glycine (G), or valine (V) then X1 is F, X2 is C and X4 is E; or Wherein when X4 is alanine (A), glycine (G), or valine (V) then X1 is F, X2 is C and X3 is S.

15. The method of claim 14, wherein SAFFLX.sub.1X.sub.2X.sub.3X.sub.4 has the amino acid sequence: a. SAFFLX.sub.1CSE, b. SAFFLFX.sub.1SE, c. SAFFLFCX.sub.1E, or d. SAFFLFCSX.sub.1, Wherein X.sub.1 is alanine (A), glycine (G), or valine (V).

16. The method of claim 15, wherein SAFFLX.sub.1X.sub.2X.sub.3X.sub.4 is mutated so that F at amino acid positions 3 and 4 is changed to S.
